(19) United States
(12) Patent Application Publication
(10) Pub. No.: US 2006/0099239 A1
(43) Pub. Date: May 11, 2006

(54) Dietary supplement for promoting removal of heavy metals from the body

(76) Inventors: Henry D. Coleman, Hastings, NY (US); R. Neil Sudol, Scarsdale, NY (US); William J. Sapone, Southport, CT (US)

Correspondence Address: WILLIAM J. SAPONE;COLEMAN SUDOL SAPONE P.C., 714 COLORADO AVENUE, BRIDGE PORT, CT, 06605, US

(21) Appl. No.: 11/312,723

(22) Filed: Dec. 20, 2005

Related U.S. Application Data
(63) Continuation-in-part of application No. 10/852,391, filed on May 24, 2004.
Application No. 10/852,391 is a continuation-in-part of application No. 10/123,576, filed on Apr. 15, 2002.

(51) Int. Cl. A61K 36/23; A61K 36/16; A61K 31/385; A61K 9/68; A61K 31/198

(52) U.S. Cl. 424/440; 424/725; 424/752; 514/565; 514/440; 424/729; 514/561; 514/562; 514/27; 514/456

(57) ABSTRACT

A dietary supplement for removing or preventing the bio-accumulation of heavy metals in the body includes one or more chelators, precursors therefore, with at least one chelator capable of crossing the blood brain barrier to capture a heavy metal ion from a site in the central nervous system, and a vascular system promoter for assisting in distributing the chelators within the body. The chelator then crosses back through the blood brain barrier with the entrained heavy metal ion. Preferably, one or more secondary chelators bind any of the heavy metal released from the primary chelator and hold it for removal via an excretion pathway. In one embodiment, the supplement includes as glutathione or metallothionine assist in moving the chelated heavy metal out into the excretion pathway. Using the dietary supplement limits the accumulation of heavy metals in the body, promotes removal of heavy metals previously accumulated in the body and thereby alleviates the symptoms and conditions associated with heavy metal toxicity.

### CROSS REFERENCE TO RELATED APPLICATION

This application is a continuation in part of application Ser. No. 10/852,391 filed May 24, 2004 which was a continuation in part of application Ser. No. 10/123,536 filed Apr. 15, 2002.

### BACKGROUND OF THE INVENTION

This invention is directed to a dietary supplement. More particularly, this invention is directed to a dietary supplement for assisting the body in cleansing itself of undesirable metals.

Mercury has been implicated in a vast array of disorders and diseases, from vascular disease to immunological malfunctions, from renal dysfunction to autism and related neurological disorders such as attention deficit disorder. Lead, arsenic and cadmium are also known to be toxic in any substantial quantities.

High levels of heavy metals such as mercury and lead are most common in people who have been exposed to high concentrations of the metals, for example, those individuals who have had the misfortune of living in a toxic waste area or near a chemical processing plant. However, even people exposed to small concentrations of a heavy metal can, over time, accumulate dangerous levels of the substance. This long term exposure has evidently occurred in children who received a series of vaccinations preserved with a mercury containing compound. Although no single vaccination likely contained enough mercury to cause any damage, the accumulation of mercury from multiple vaccinations over a two or three year period resulted in dangerous levels of mercury in a significant percentage of children. Women also may accumulate aluminum compounds from daily use of certain antiperspirants, speculated as being a link with an increase in breast cancer.

It is known that mercury toxicity can disrupt the immune system, and may be implicated in auto-immune disease, and many food and environmental allergies may be attributed to sensitivity caused by mercury exposure. It is also known that the effects of mercury in the body can include behavioral changes, depression, confusion, irritability and hyperactivity, as well as fatigue, insomnia, slurred speech, etc.

It does not appear to be a coincidence that these symptoms correlate with many childhood conditions that are approaching near epidemic proportions. For example, Attention Deficit Disorder (ADD) and Attention Deficit/Hyperactivity Disorder (ADHD) (severally and collectively hereinafter referred to as “AD”) are developmental disorders of self-control. They consist of problems with attention span, impulse control and activity level. These problems are reflected in impairment of a person's will or capacity to control his or her own behavior relative to the passage of time and to keep future goals and consequences in mind. The number of children having AD and related conditions, as well as allergies, appears to have grown in parallel with the level of mercury exposure through vaccines and otherwise. At present there are various drug treatments used to address these conditions, but these are directed to alleviating symptoms and are not directed to removing a possible source of the condition.

On the cellular level, mercury appears to inhibit the natural action of enzyme systems, to depolarize cell membranes, to increase intracellular calcium, to alter neurotransmitter release and inhibition. Impact on the neurological system, digestive tract and immune system are implicated.

Virtually everyone who lives in today's society, consuming modern goods and living in an environment polluted with decades of heavy industrial output, can be expected to take in significant quantities of heavy metals over a lifetime. Mercury can leach out of dental fillings, at least when the fillings are first created, are worked on by a dentist or are subject to considerable fatigue stressing. Aluminum, a lighter but still highly reactive metal to be sure, but not a mineral found in natural organic tissues, can be absorbed from aluminum in cans, foils, cookware and cosmetics such as antiperspirants. There has been a link suggested between use of such antiperspirants with breast cancer. Long term intake of even trace quantities of heavy metals can produce or exacerbate debilitating illness, especially neurological disease states and conditions. Where the heavy metals are absorbed in the central nervous system, various neural diseases including neuro-degenerative diseases may result, including, for instance, Alzheimer's, Parkinson's and related diseases. In children, heavy metal toxicity (especially lead and mercury, but also cadmium and arsenic, among others) is implicated in speech impediments, learning disabilities, attention deficit disorder (ADD) and attention deficit hyperactivity disorder (ADHD), autism, autism spectrum diseases and related developmental diseases.

There are medical treatments for acute heavy metal toxicity, relying on the use of strong chelating agents such as DMSA. However, such treatments are not available to those exhibiting less dramatic but more insidious effects of low levels of mercury toxicity caused by bio-accumulation, and such medical treatments cannot be used as a prophylaxis to limit bio-absorption of heavy metals or to support the natural bodily processes for sequestering and removing heavy metal contaminants.

Any composition or method that provides a means to reduce heavy metal levels, using natural ingredients such as specific dietary supplements, would be quite useful, particularly for those who do not need therapeutic intervention but rather who wish to actively take steps to maintain their health before such intervention is called for.

Accordingly, people who wish to protect themselves from the risks associated with exposure to heavy metals by enhancing removal of these materials from the body have a need for products to assist the body in dealing with such heavy metals and for supporting the body's natural restorative functions.

### OBJECTS OF THE INVENTION

An object of the present invention is to provide a dietary supplement for assisting the body in performing natural cleansing or detoxification functions.

A more specific object of the present invention is to provide a dietary supplement for use in assisting the body in the removal of heavy metals such as nickel, lead, mercury, arsenic, cadmium, aluminum and tin.

Another object of the present invention is to provide such a dietary supplement which assists in not only removing heavy metals from the body but also assists in reversing the effects of heavy metal toxicity.

It is still another object of the present invention to provide a method for reducing or removing metals in individuals who may be at risk for or show symptoms of heavy metal toxicity which may manifest as Alzheimer's disease, Parkinson's disease, learning disabilities, autism, ADD, ADHD, speech disabilities and related neurological conditions, as well as chronic fatigue syndrome, sleeplessness, depression, anxiety, bipolar disease, multiple sclerosis, allergies, cardiovascular disease, cancer and other diseases and conditions where heavy metal toxicity in a patient may be implicated.

These and other objects of the present invention will be apparent from the drawings and descriptions herein. Although every object is attained by at least one embodiment of the invention, there is not necessarily any embodiment in which all of the objects of the invention are achieved.

### SUMMARY OF THE INVENTION

The present invention is directed to dietary supplements to be taken regularly, preferably at least once daily and more preferably at least twice or three times daily for purposes of assisting natural cleansing mechanisms to remove heavy metals from the body. In particular, the dietary supplements are intended to assist in the removal of heavy metals from the individual, and in particular, the central nervous system, immune system, skeletal system, especially in the case of lead and musculature.

Dietary supplements in accordance with the present invention are intended for long term use and are administrable from once up to six times daily for bolstering the body's natural defenses against heavy metals. The dietary supplements are preferably administered at least twice daily. The amounts of the various components are relatively small and fall within acceptable limits which are now included in a number of dietary supplements unrelated to the present invention. When used on a consistent/continual basis, the supplements serve to assist the body's natural mechanisms for the removal of toxic metals. The dietary supplements are particularly effective for the removal of mercury and lead. The supplements are also at least partially effective for assisting in the removal of other heavy metals such as nickel, cadmium, aluminum, arsenic, and tin.

A dietary supplement in accordance with the present invention includes at least one primary natural chelator which is able to cross the blood brain barrier with an entrained (chelated) heavy metal atom. The supplement also includes at least one secondary chelator or a precursor of the secondary chelator. The secondary chelator is able to move the heavy metal through the body away from the central nervous system. This secondary chelator may function to accept the heavy metal from the primary chelator. Alternatively or additionally, the primary or secondary chelator may function to move the chelated heavy metal out into an excretion pathway.

A vascular system promoter is also included in the inventive composition. The vascular system promoter increases blood flow to enhance distribution in the body of the chelators so as to enhance effectiveness.

The primary chelator is provided in the dietary supplement in an amount effective to move a selected heavy metal species from a user's central nervous system into the user's vascular system for subsequent release from the body via an excretion pathway. The secondary chelator functions to accept or chelate the heavy metal released from the primary chelator. The secondary chelator is present in an amount effective to capture the heavy metal species from the primary chelator to effectively prevent recycling of the heavy metal species back into the central nervous system. Of course, some recycling may occur, but the secondary chelator serves to prevent a recycling of all of the captured metal ions and thus enables the body to cleanse itself of the target heavy metal. Through long term usage of the inventive composition, substantial progress can be made in eliminating such heavy metals from the body, which also function as a propylactic to capture such heavy metal species as are encountered through normal environmental interaction, such as present in trace amounts in food, water and air, so as to prevent a bioaccumulation of such metals with aging.

Where the secondary chelator functions to move the chelated heavy metal out into an excretion pathway, the dietary supplement may incorporate a precursor of the secondary chelator rather than the chelator itself. In this case, the secondary chelator may be glutathione or metallothionine and the precursor is respectively glycine, cysteine, N-acetylcysteine or methionine or glutathione or glutamic acid, preferably glycine or methionine, more preferably methionine.

A dietary supplement in accordance with another embodiment of the present invention may include a primary chelator, a secondary chelator, a vascular system promoter, and a tertiary chelator or a precursor of a tertiary chelator. In this case, the primary chelator crosses the blood brain barrier to capture a heavy metal ion from a site in the central nervous system. The primary chelator then crosses back through the blood brain barrier with the entrained heavy metal atom. The secondary chelator acquires the heavy metal from the primary chelator in the blood or other site outside of the central nervous system and may transfer the metal to the tertiary chelator such as glutathione or metallothionine which moves the chelated heavy metal out into an excretion pathway. Again, the precursor of the tertiary chelator is, for example, glycine or methionine. It is to be noted that the secondary chelator may also function to some extent to remove heavy metal species directly from the tissues of the central nervous system.

In this embodiment of a dietary supplement in accordance with the present invention, the primary chelator may be alpha lipoic acid (thiooctic acid), while the secondary chelator may take the form of a bioflavonoid, preferably a bioflavonoid such as quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside), citrus bioflavanoid complex and lemon bioflavanoid complex, among others. In addition, other chelators unrelated to bioflavonoids may also be used in particular, cyanadin, cyanadin chloride, esculetin and tannins, including caffeic acid. Alpha lipoic acid in its reduced or oxidized form, preferably, its reduced form, is provided in the dietary supplement in an amount sufficient to move a selected heavy metal species from a user's central nervous system into the user's vascular system. The bioflavonoid is present in an amount sufficient to capture amounts of the heavy metal species from the alpha lipoic acid to effectively prevent recycling of the heavy metal species back into the central nervous system. The bioflavonoid thus serves at least in part to prevent a recycling of all of the captured metal ions and thus enables the body to cleanse itself of the target heavy metal. The bioflavonoid may also serve as a primary chelator as certain bioflavonoids may cross the blood brain barrier or the lipid membrane of neuronal cells and chelate metals within lipid bilayers of the cell membrane or other lipid cellular structures.

Pursuant to another feature of the present invention, a dietary supplement for assisting the body's natural cleansing mechanisms for removing toxic metals may further comprise (optionally) a mineral in an amount sufficient to replace the selected heavy metal species removed from the central nervous system, the mineral being a natural component of cellular processes and biochemical structures of the body, especially the central nervous system. The replacement mineral is preferably taken from the group consisting of calcium, magnesium, zinc and mixtures thereof and may optionally include additional minerals such as molybdenum, manganese, chromium, boron, copper, iron, selenium, vanadium and mixtures thereof. This feature of the present invention is based on the recognition that heavy metals are toxic in part because they replace other metal species or minerals that naturally occur in cellular and molecular structures of the body. In replacing, for example, calcium, magnesium and zinc in cell membranes, enzymes, other cellular substructures, etc., the heavy metals, for example, nickel, lead, mercury, arsenic, cadmium, and aluminum, often prevent the proper functioning of those cellular and molecular structures. Natural physiological processes are impaired, blocked, or subverted resulting in damage to the individual's normal psychological, physiological, mental, linguistic, and social functioning. Memory is often impaired.

The inclusion of such minerals in a dietary supplement assists the body in replacing the captured heavy metal species with the minerals which were ousted by the heavy metals originally.

Pursuant to a further feature of the present invention, a dietary supplement for assisting the body's natural cleansing mechanisms for removing toxic metals may further comprise at least one vitamin selected from the group consisting of thiamine (vitamin B1), vitamin B6, folic acid, vitamin B12, vitamin A, vitamin E, and vitamin C. The roles of antioxidants are well known. The inclusion of vitamins and antioxidants assists the body in repairing tissues on a molecular level and preventing further damage by heavy metal incursions.

In accordance with an additional feature of the present invention, a dietary supplement may further comprise cilantro or coriander or an extract (water, water/alcohol, especially water/ethanol, ethanol, isopropanol or ether extract) of cilantro or coriander. Soup with Chinese parsley, also known as cilantro has been reported as being capable of removing heavy metals. In the inventive composition, a liquid extract of Cilantro or a dried powder extract of Cilantro is used as a primary chelator, preferably in combination with another primary chelator such as alpha lipoic acid.

A dietary composition in accordance with another embodiment of the present invention has blend containing a cilantro extract in combination with a primary chelator (preferably in the form of a bioflavonoid) selected from the group consisting of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside, citrus bioflavanoid complex and lemon bioflavanoid complex, among others. In addition, other chelators unrelated to bioflavonoids may also be used in particular, cyanadin, cyanadin chloride, esculetin and tannins, including caffeic acid, and a precursor of a secondary chelator. The secondary chelator may be glutathione or metallothionine. In that case, the precursor is glycine or methionine, respectively.

The blend is included in an amount which is effective to capture a heavy metal species such as mercury or lead from body tissues, which exemplarily include nervous system tissues. The precursor is added to the blend in an amount which generates quantities of the secondary chelator effective to transfer or capture from the primary chelator for removal through to the heavy metal species captured by the primary chelator for removal through an excretion pathway. The heavy metal species may be acquired by the secondary chelator throughout the body, although it is expected that most of this acquisition will occur in the vascular system.

The vascular system promoter may be selected from a number of nutraceuticals that have positive effects on blood flow. For example, L-arginine is believed to enhance vascular flow and blood distribution, Rhodeola Rosea extract similarly is believed to improve blood flow, and citrulline and Ginkgo Biloba may also provide beneficial effects. Generally, those nutraceuticals known for promoting endurance, oxygen availability or vascular flow or distribution would be beneficial in the composition of the present invention.

Another embodiment of a dietary supplement composition in accordance with the present invention includes (a) at least one primary chelator in an amount sufficient to move a selected heavy metal species from a user's central nervous system into the user's vascular system and (b) a secondary chelator in an amount sufficient to capture amounts of the heavy metal species from the primary chelator to effectively prevent recycling of the heavy metal species back into the central nervous system. In this embodiment, the primary chelator may be alpha lipoic acid and/or the cilantro extract, while the secondary chelator is preferably a lipophilic chelator selected from the groups consisting of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside, citrus bioflavanoid complex and lemon bioflavanoid complex, among others. In addition, other chelators unrelated to bioflavonoids may also be used in particular, cyanadin, cyanadin chloride, esculetin and tannins, including caffeic acid. Alternatively, the primary chelator may be one or more of the above described secondary chelators, preferably a mixture of the above described secondary chelators, inasmuch as the mixture may function as both a primary and secondary chelator. The bioflavonoids selected from the group consisting of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside), citrus bioflavanoid complex and lemon bioflavanoid complex, among others are preferred in this regard, with quercetin, rhamnetin, fisetin, kaempferol, rutin, quercitrin, hyperosid, cyanadin and caffei acid clearly preferred. Alternatively, gingko biloba extract which contains several of these compounds may be used. In addition, this embodiment may also comprise a precursor in an amount effective to stimulate or increase production in the user's body of a tertiary chelator able to transfer the captured heavy metal species from the secondary chelator into an excretion pathway. The tertiary chelator may be glutathione or metallothionine, with the precursor being glycine, methionine, cysteine, n-acetyl cysteine, glutamic acid, glutamine or a salt therof.

Any one or more of the compositions according to the present invention may further comprise at least one mineral such as calcium, magnesium or zinc in an amount effective to replace the selected heavy metal species removed from the central nervous system, the mineral being a natural component of cellular molecular structure of the central nervous system. Other minerals selected from the group consisting of molybdenum, manganese, chromium, boron, copper, iron, selenium, vanadium and mixtures thereof may also be included in compositions according to the present invention. This embodiment optionally includes at least one vitamin taken from the group consisting of thiamine, vitamin B6, folic acid, vitamin B12, vitamin A, vitamin E, and vitamin C. Cilantro or coriander or extract thereof may be included also.

A dietary supplement composition for assisting the natural body functions in sequestering and removing, or “cleansing” the body of heavy metals may specifically comprise, in accordance with the present invention, alpha lipoic acid in an amount of about 10 mg to about 500 mg (preferably within the range of about 25 mg to about 100 mg), and least one chelating compound taken from the group consisting of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside), citrus bioflavanoid complex and lemon bioflavanoid complex, among other compounds, including cyanadin, cyanadin chloride, esculetin and tannins, including caffeic acid in amount ranging from about 5 mg to about 1 gram, preferably about 10 mg. to about 500 mg. or gingko biloba in comparable quantities as a substitute therefore. In addition, the composition may include at least one amino acid taken from the group consisting of glycine and methionine, in an amount of about 50 mg to about 500 mg. Where the chelating compound is selected from the group consisting of quercetin, dihydroquercetin, rutin, quercitrin, hyperosid, cyanadin, esculetin, caffeic acid, citrus bioflavonoids, lemon bioflavonoids or mixtures thereof, the composition optionally comprises at least one additional bioflavonoid preferably taken from the group consisting of catechin, epicatechin, rhamnetin, fisetin, dihydrofisetin, kaempferol, robinin, 3-hydroxyflavone, and mixtures thereof in an amount of about 5 mg to about 500 mg., preferably about 10 mg to about 300 mg.

The vascular system promoter may be selected from one or more of L-arginine, Rhodeola Rosea extract, green tea extract, Ginkgo Biloba extract, etc. These may be present at from about 10 mg-300 mg per dose.

The dietary supplement composition may further comprise a mineral taken from the group consisting of calcium, magnesium, and zinc, preferably in an amount of about 2.5 mg to about 500 mg. (preferably, with the amount of zinc if used being at the lower end of the range and preferably calcium if used being at the higher end of the range), and at least one vitamin taken from the group consisting of thiamine, vitamin B6, folic acid, vitamin B12, vitamin A, vitamin E, and vitamin C.

The present invention recognizes that heavy metals are present in the natural environment (air, earth, water) and in a host of consumer products. The metals absorbed by the body in trace amounts over the short term accumulate to the eventual detriment of the individual's health, unless the diet provides the individual with the suitable natural components or materials to capture, eject or allow the body's natural elimination processes to expunge the heavy metals from the body. The dietary supplements of the present invention provide a relatively safe means, using natural ingredients with little or no side effects, by which users may cleanse themselves of heavy metals accumulated over years and limit further bio-accumulation of heavy metals going forward. Thus the invention is both a restorative treatment and prophylaxis to limit detrimental effects from continued exposure to trace quantities of such heavy metals.

### DEFINITIONS

The term “heavy metal species,” “heavy metal,” “heavy metal atom,” and “heavy metal ion” are used interchangeably herein to designate atoms and cations of those metals which are essentially toxic to human beings. Such toxic metals generally do not naturally occur in any significant quantities in human beings and when present in elevated quantities are likely to result in impairment of normal human functioning. Such impairment may affect short term and long term memory, linguistic abilities, social skills, motor skills, cognition and other basic capabilities. Heavy metals typically include mercury, lead, nickel, arsenic, cadmium, aluminum, some species of chromium, and tin with implications for the different heavy metals in different conditions, disease states and symptomology.

The word “chelator” as used herein refers to a chemical substance which has a relatively high affinity for at least one heavy metal species. This affinity is such that the chelator is able to capture or complex the heavy metal ions or atoms from other molecules, such as lipids, proteins, enzymes, other chelators, etc., and maintain a sufficient hold on the captured metal to move the metal from the capture site. A chelator may function primarily to move heavy metals from the central nervous system or other organic tissues into the vascular system. Alternatively, a chelator may function chiefly to move captured metal ions through and out of the vascular system. Alternatively again, a chelator may function mainly to move captured metal ions into an excretion pathway. A chelator may have a pincer-type structure or moiety with two or more opposed jaws formed by chemical groups having a negative charge or negative character, for instance, sulfhydryl groups, ketone groups, carboxy groups, hydroxyl groups. The groups are spaced from one another by distances facilitating the capture of heavy metal ions. In certain instances, the chelator may also have anti-oxidant properties or other properties in addition to its chelating characteristics.

The term “primary chelator” is used herein in a general sense to denote a chelator which functions mainly to capture heavy metal species from tissues, cells, and molecules such as enzymes in the human body. The term “primary chelator” is used herein in a specific or narrow sense to denote a chelator which functions mainly to capture heavy metal species from the central nervous system.

The term “secondary chelator” as used herein denotes a chelator which functions to accept captured heavy metal species released from a primary chelator and to move the metal further along a removal pathway through a patient tissues and organ systems. A secondary chelator may also function as an additional primary chelator in capturing one or more types of heavy metals from a person's tissues (CNS, muscle, connective, bone, visceral tissues, etc.) and molecules (enzymes, antigens, antibodies, fatty acids, lipids, etc.).

The term “vascular system promoter as used herein denotes an ingredient that has a positive effect on blood flow or distribution in the body, to enhance the effectiveness of the chelating compounds circulating through the body, as well as assisting in promoting removal from the body.

The word “effective” when used herein is described in relation to the action of a dietary supplement component intended to be regularly consumed in relatively small amounts over long periods of time. The effectiveness of a supplement component is thus determined with reference to this intended use.

The term “excretion pathway” as used herein denotes any of the various routes by which the body rids itself of toxins. The three principal excretion pathways are through the kidneys (urinary), intestines (biliary), and sweat glands.

### DETAILED DESCRIPTION OF THE INVENTION

The dietary supplements described are best suited to long term use and are preferably to be taken regularly, at periodic intervals. The supplements are administrable from once up to six times daily for supplementing and bolstering the body's natural defenses against heavy metals. A preferred schedule is twice daily. However, higher rates of consumption are certainly acceptable. As the consumption rate increases, the amounts of the individual components should be decreased.

The compositions described herein are intended as additions to normal diet and should not be considered as substitutes for proper nutrition. It is recommended that the supplements be taken at mealtime to take advantage of the full panoply of nutritive constituents of traditional foods. Used in this way, the compositions described herein are best able to promote and supplement the natural cleansing mechanisms of the body and to assist in the removal of heavy metals.

For assisting the natural metabolic processes of the body in cleansing the central nervous system of heavy metals, a dietary supplement includes at least one primary chelator which is able to cross the blood brain barrier with an entrained heavy metal atom. Alpha lipoic acid is known to be such a chelator. This may be combined with an extract of cilantro in a primary chelator blend. The supplement also includes at least one secondary chelator or a precursor of the secondary chelator. The secondary chelator is able to move the heavy metal through the body away from the central nervous system. This secondary chelator may function to accept the heavy metal from the primary chelator. Alternatively or additionally, the secondary chelator may function to move the chelated heavy metal out into an excretion pathway.

Quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside), citrus bioflavanoid complex and lemon bioflavanoid complex, cyanadin, cyanadin chloride, esculetin and tannins, including caffeic acid are secondary chelators of the first type. Quercetin, rutin, quercitrin and hyperosid are preferred secondary chelators, and may administered as a gingko biloba extract as a substitute therefore as such extract typically contains quercetin, kaempferol and rhamnetin. In the present invention, the secondary chelator acquires captured heavy metal cations from a primary chelator such as alpha lipoic acid and transports the captured heavy metal further away from the central nervous system. It is believed that quercetin, as well as other lipophilic secondary chelators may additionally function as a primary chelator, to extract heavy metals from natural organic tissues (nerve, bone, muscle, connective, cardiovascular, visceral, pulmonary, etc.) and physiological molecules (enzymes, antigens, molecular pumps, lipids, fats, etc.), especially those within the cell membrane or other lipophilic cellular structures. The inclusion of a secondary chelator such as quercetin and/or one or more other lipophilic secondary chelators serves to prevent the primary chelator from recycling the captured heavy metal cations back into the central nervous system (“CNS”). One or more secondary chelators thus tip the equilibrium balance of the primary chelator/heavy metal system away from the CNS.

The primary chelator, e.g., alpha lipoic acid or a bioflavonoid or related lipophilic secondary chelator such as quercetin, is provided in the dietary supplement in an amount sufficient to move effective amounts of a selected heavy metal species from a user's central nervous system into the user's vascular system. Where the secondary chelator functions to accept the heavy metal from the primary chelator, the secondary chelator is present in an amount sufficient to capture amounts of the heavy metal species from the primary chelator to effectively reduce or prevent recycling of the heavy metal species back into the central nervous system. Of course, some recycling may occur, but the secondary chelator serves to prevent a recycling of all of the captured metal ions and thus enables the body to gradually cleanse itself of the target heavy metal.

Glutathione or metallothionine may be included in the dietary supplement as a secondary chelator acquiring captured metal cations either from a primary or another secondary chelator and moves the chelated heavy metal away from the central nervous system out into an excretion pathway. However, glutathione and metallothionine are generally broken down during the digestive process by digestive enzymes and relatively little of these agents may be delivered efficiently orally. Accordingly, to provide effective amounts of such a secondary chelator, the dietary supplement incorporates a precursor of the respective secondary chelator rather than the chelator itself. The precursor is used by the body to generate the secondary chelator, e.g., glutathione or metallothionine. Thus, instead of glutathione or metallothionine, the supplement includes a precursor in the form of glycine, cysteine, n-acetyl cysteine, S-adenosyl methionine and/or methionine, preferably, glycine and/or methionine.

Another dietary supplement formulation includes a primary chelator, a secondary chelator, and a tertiary chelator or a precursor of a tertiary chelator. In this case, the primary chelator (e.g., alpha lipoic acid or other lipophilic chelator, preferably such as quercetin) crosses the blood brain barrier to capture a heavy metal ion from a site in the central nervous system. The primary chelator then crosses back through the blood brain barrier with the entrained heavy metal atom. The secondary chelator (as described hereinabove) acquires the heavy metal from the primary chelator in the blood or other site outside of the central nervous system and transfers the metal to the tertiary chelator (glutathione or metallothionine) which moves the chelated heavy metal out into an excretion pathway. Again, the precursor of the tertiary chelator is for example glycine, methionine, cysteine, acetyl L cysteine, glutamic acid, glutamine or a salt thereof. It is to be noted that the secondary chelator may also function to some extent to remove heavy metal species directly from the tissues of the central nervous system or alternatively, as antioxidants.

Alpha lipoic acid is provided in the dietary supplement in an amount sufficient to move a heavy metal species (particularly lead or mercury) from a user's central nervous system into the user's vascular system. The secondary chelators as described herein (preferably, the bioflavonoids) are present in amounts sufficient to capture amounts of the heavy metal species from the alpha lipoic acid to effectively prevent recycling of the heavy metal species back into the central nervous system. The secondary chelators thus serve at least in part to prevent a recycling of all of the captured metal ions and thus enables the body to cleanse itself of the target heavy metal.

The vascular promoter is incorporated in the inventive composition in an amount sufficient to enhance distribution of the chelator compounds throughout the body. Not only should chelators be present at minimal levels to support the body in capturing the heavy metals, but increased blood flow and distribution can assure better bioavailability of these compounds in the body, and assure more rapid removal once a metal species is captured, and quicked repair/replacement with beneficial compounds such as calcium or magnesium.

The amounts of the chelating components of a dietary supplement composition as described herein are small relative to the amounts of the same components in other kinds of dietary supplements. Generally, the primary and secondary chelators are included in amounts of about 0.05 to about 10 milligrams per kilogram of body weight, more preferably about 0.1 to about 3.5 milligrams per kilogram of body weight, even more preferably about 0.5 to about 2.5 milligrams per kilogram of body weight. Amounts in the lower weight range are preferred where the rate of supplement consumption is high, for instance, five or six times daily, and/or where the purpose of consumption is prophylactic, i.e., to remove the heavy metals as they are acquired, rather than to remove heavy metals which have accumulated over a long period. Conversely, amounts in the higher weight range are preferred where the rate of supplement consumption is low, for instance, one or two times daily, and/or where the purpose of consumption is to cleanse the body of accumulations of heavy metals incurred over a long period or from exposure to unusually high concentrations of the toxic substances. Such a high concentration may occur, for example, when a person has lived for a substantial period near, or in a waste runoff region of, a manufacturing plant using or producing heavy metals.

It appears, at least in certain instances, that heavy metals inadvertently admitted into the body become attached at locations normally occupied by other minerals of a lower atomic weight such as calcium, magnesium and zinc. The replacement of these natural minerals with heavy metal atoms is likely to prevent the proper functioning of the tissues or molecules to which the heavy metal is attached. When the heavy metals are extracted by a primary chelator, as described herein, the vacated positions are desirably filled by lower-weight minerals such as calcium, magnesium or zinc. Accordingly, a dietary supplement as described herein preferably includes at least one mineral in an amount (5 to 1000 mg) effective to replace the selected heavy metal species removed from the central nervous system. The selected mineral, for instance, calcium, magnesium, and/or zinc, is a natural component of cellular molecular structure. These minerals are necessary dietary components and may be included in large amounts to optimize the chances that the sites vacated by captured heavy metal species are promptly filled by an appropriate substitute atom. The inclusion of the minerals in a dietary supplement thus facilitates a natural healing process by providing the substitute minerals at the precise time they are needed.

Another, optional active component of a dietary supplement for assisting the body's natural cleansing mechanisms for removing toxic metals is a vitamin or antioxidant taken from the group consisting of thiamine, vitamin B6, folic acid, vitamin B12, vitamin A, vitamin E, and vitamin C. The roles of vitamins and antioxidants are well known. The inclusion of vitamins and/or antioxidants assists the body in repairing tissues on a molecular level and preventing further damage by heavy metal incursions. These vitamins and/or antioxidants may be included in any desirable combination and in amounts customary in the trade.

In accordance with the present invention, the inventive composition preferably contains a cilantro or coriander extract. This may be liquid or a dried powder, for example it may be a hot water extract, dried with a carrier, comprising 10-50:1 carrier to extract, more preferably about 25:1. This dried extract is preferred so as to improve ease in creating specific blends in accordance with the present invention.

A typical cilantro extract may be obtained by various processes known for making extractions from herbs, such as water or water/alcohol extraction processes, supercritical CO₂ extraction, etc. Typically such an extract contains linahols and glucosides, such as various β-D-glucopyranosides. Long claim (C6-C10) alcohols and aldehydes are common and it may also contain phospholipids, phytosterols and phenols. Such an extract can function as a primary or secondary chelator for mercury, as well as be used to prepare a primary chelator blend.

A dietary supplement composition for assisting the body in cleansing itself of toxic metals may have a primary chelator in the form of at least one compound selected from the group consisting of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside, citrus bioflavanoid complex, lemon bioflavanoid complex, cyanadin, cyanadin chloride, esculetin and tannins, including caffeic acid and a precursor of a secondary chelator. Where the secondary chelator is glutathione or metallothionine, the precursor is glycine or methionine, respectively. The primary chelator is included in an amount which is effective to capture a heavy metal species such as mercury or lead from body tissues, especially lipophilic body tissues which exemplarily include nervous system tissues. The precursor is provided in an amount which generates quantities of the secondary chelator effective to transfer, from the primary chelator to an excretion pathway, the heavy metal species captured by the primary chelator. The heavy metal species may be acquired by the secondary chelator throughout the body, although it is expected that most of this acquisition will occur in the vascular system.

Another embodiment of a dietary supplement composition comprises (1) at least one primary chelator (alpha lipoic acid, quercetin, quercitran, or gingko biloba, as a substitute therefor rutin, hyperosid, rhamnetin, cyanadin, fisetin) in an amount sufficient to move a selected heavy metal species from a user's central nervous system into the user's vascular system and (2) a secondary chelator (at least one of quercetin, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhamnoside), hyperosid (quercetin-3-D-galactoside), robinin (kaempferol-3-rabinosie-7-L-rhamnoside, citrus bioflavanoid complex, lemon bioflavanoid complex, cyanadin, cyanadin chloride, esculetin and tannins, including caffeic acid) in an amount sufficient to capture amounts of the heavy metal species from the primary chelator to effectively prevent recycling of the heavy metal species back into the central nervous system. In addition, this embodiment may also comprise a precursor (glycine, methionine) in an amount effective to stimulate or increase production in the user's body of a tertiary chelator (glutathione, metallothionine) able to transfer the captured heavy metal species from the secondary chelator into an excretion pathway. This embodiment may further comprise a mineral such as calcium, magnesium or zinc in an amount (2.5 to 1000 mg, 5 to 500 mg preferably) effective to replace the selected heavy metal species removed from the central nervous system. This embodiment optionally includes at least one vitamin or antioxidant taken from the group consisting of thiamine, vitamin B6, folic acid, vitamin B12, vitamin A, vitamin E, and vitamin C. Cilantro or coriander, including an extract thereof may be included also.

Compositions according to the present invention may be formulated for delivery to a mammal, preferably a human. The compounds of this invention may be incorporated into formulations for all routes of administration including for example, oral and parenteral including intravenous, intramuscular, intraperitoneal, intrabuccal, transdermal and in suppository form.

For administration, the compositions may be formulated into forms suitable for the above sources of administration, for example, suitable forms of oral administration such as tablets, granules, powders, coated tablets, hard capsules, soft capsules, liquids, suspensions and gels.

One particular novel form of administration is by inclusion in confectionary items. In particular, the presence of heavy metals is not limited to adults and in fact is likely most pronounced in children who have been exposed to such heavy metals, by vaccination, ingestion of lead paints, etc. While the above compositions can be administered by way of tablets or capsules, for example, such forms are not well received by children. Further the compositions of the invention are best administered over time to allow the body's natural mechanisms to remove such substances from the system, and long term consistent compliance, with children in particular, is difficult to achieve if the form of administration is resisted.

Consequently, the compositions of the present invention may be formulated for oral administration in a tablet, capsule, gelatin capsule, tincture, suspension, powder, granular or other form or as a component of a food, beverage or confectionary, such as being incorporated into soft gummy candy (i.e. gummy bears, worms, etc.), hard candy or gum balls, among others.

In a preferred embodiment, the primary chelator and secondary chelator are combined and integrated into a gummy candy formulation, to produce gummy candies, each of which delivers all or a portion of the proper dosage of chelating ingredients. Preferably these are prepared to contain, for example 1/20th to ⅕th, preferably about 1/10th the daily adult dose per candy piece, such that the weight appropriate amount can be administered to a child. For example while a 2501b adult might need to consume 10 gummy candies, a 501b child may need to consume only 2, and in this way the appropriate amount, based on weight can easily be determined.

Further, the various components of the inventive formulation can be segregated into different gummy candies, to allow further tailoring to individual needs. Thus the primary and secondary chelator could be in one gummy, a tertiary chelator in another, various vitamins and replacement minerals in another, etc, so that a child can eat 4-6 pieces per day and receive the full compliment of chelating compounds, antioxidants, and also mineral replacement. Use of such a delivery system may be very important to achieving long term support of the bodies natural system for eliminating contaminants and heavy metals, and to actively prevent further bioaccumulation.

Nutritional supplement compositions based upon these novel chemical components comprise the above-described components in an effective amount for removing heavy metals from a mammal, especially including a human. One of ordinary skill in the art will recognize that an effective amount of one of more components to be included in the present compositions will vary with the conditions to be resolved and the heavy metal which is to be removed from the individual, as well as the pharmacokinetics of the agent used, and the condition of the patient (animal or human) treated.

The preferred route of administration is by an oral route. Compositions according to the present invention are formulated preferably in admixture with a pharmaceutically acceptable carrier. In general, it is preferable to administer the pharmaceutical composition in orally-administrable form, but a number of formulations may be administered via a parenteral, transdermal, buccal, subcutaneous, suppository or other route. Of course, one of ordinary skill in the art may modify the formulations within the teachings of the specification to provide numerous formulations for a particular route of administration without rendering the compositions of the present invention unstable or compromising their therapeutic activity. In particular, the modification of the present compounds to render them more soluble in water or other vehicle, for example, may be easily accomplished by minor modifications to the formulations, which are well within the ordinary skill in the art. It is also well within the ordinary skill to modify the route of administration and dosage regimen of a particular component in order to manage the pharmacokinetics of the present compositions for maximum beneficial effect to the patient, for example, including integration with confectionaries, food or beverages.

Adjuvants normally used in formulating nutritional supplements may be used in formulating the present invention as carriers, such as syrup, gum Arabic, gelatin, sorbitol, polyvinyl pyrrolidone, magnesium stearate, talc, polyethylene glycol, silica, lactose, sucrose, corn starch, calcium phosphate, starch, carboxymethyl cellulose, water, ethanol, glycerol, mannitol, among others. Optional ingredients such as coloring agents, flavors, dissolution acids, suspending agents, dispensing agents, etc., may also be used. The composition may be formulated to provide delayed or controlled release, using enteric coatings, micro-encapsulation or other techniques known in the art. Of course, for the confectionary form of the invention, any of the components typically used to produce such confectionary items may be used as a base or carrier for delivering the compounds of the invention, adapted, if necessary to avoid detrimental interactions during production.

An effective amount of the composition represents an amount necessary to remove, prevent or limit further bio-accumulation of a heavy metal in the body. The compounds described are effective over a wide range of dosages, and it is understood that the dosage many vary based on the symptom to be treated, its severity, the age, weight and response of the individual person, and the chosen route of administration.

Treating in accordance with the present invention may include prophylaxis of heavy metal accumulation, or of a specific symptom, amelioration, elimination, or attenuation of the condition or symptom related to heavy metal toxicity due especially to low level exposure to such heavy metals in the environment.

### EXAMPLES

The following exemplary formulations set forth active ingredients of dietary supplements for assisting the body to carry out natural cleansing processes to rid itself of heavy metal interlopers. Accordingly, each formulation may be used with fillers, buffers, binders, etc., normally found in dietary supplements. Preferably, the nonactive ingredients are selected on the basis of their known biocompatibility with the human organism. For instance, possible allergenic substances are to be preferably avoided. Where the compositions are intended for use in children, flavors and food ingredients may be added. Alternatively, the examples below may be implemented as additives to candy, cookies, ice creams and other foods of interest to children. In that event, the amounts of the active ingredients listed in the below examples are selected with due consideration to the amounts of the foods children might be expected to consume in a given (daily) period.

The compositions may be formulated through traditional pharmaceutical compounding procedures and other procedures which are well known in the art. The compositions may be produced in tablet form, gums, oral suspensions, capsules including hard and soft gelatin capsules, among others.

In the examples below, a slash mark (“/”) means that the compounds on opposite sides may be included alternatively in the amount indicated or together in the same weight amount. Note that the individual components are generally weighed out and thoroughly mixed, either as solids or liquids.

### Specific Formula Examples

### Example 1

### Basic Composition

| | |
|---|---|
| Cilantro Extract (25:1) | 400 mg |
| Gingko Biloba Extract (24/6) | 40 mg |
| Rhodeola Rosea Extract | 140 mg |
| (0.8% salidrosides, 0.8% rosavins) | |
| Alpha lipoic acid | 40 mg |
| L-Arginine | 100 mg |
| Vitamin C | 40 mg |
| Quercetin | 15 mg |
| Glycine/methionine | 75 mg |

### Example 2

### Mineral Enhanced

| | |
|---|---|
| Cilantro Extract (25:1) | 400 mg |
| Gingko Biloba Extract (24/6) | 40 mg |
| Rhodeola Rosea Extract | 140 mg |
| (0.8% salidrosides, 0.8% rosavins) | |
| Alpha lipoic acid | 40 mg |
| L-Arginine | 100 mg |
| Green Tea Extract | 100 mg |
| (80% polyphenols, 60% catechins, 30% EGCG) | |
| Vitamin C | 40 mg |
| Glycine/methionine (50/50) | 75 mg |
| Calcium (as gluconate) | 100 mg |
| Magnesium(as citrate) | 50 mg |
| Zinc(as citrate) | 10 mg |

### Example 3

### Further Enhanced Formula

| | |
|---|---|
| Cilantro Extract (25:1) | 400 mg |
| Gingko Biloba Extract (24/6) | 40 mg |
| Rhodeola Rosea Extract | 140 mg |
| (0.8% salidrosides, 0.8% rosavins) | |
| Alpha lipoic acid | 40 mg |
| L-Arginine | 100 mg |
| Quercetin | 15 mg |
| Glycine/methionine | 250 mg |
| Calcium | 100 mg |
| Magnesium | 50 mg |
| Zinc | 15 mg |
| Thiamine | 50 mg |
| Vitamin B6 | 50 mg |
| Folic acid | 50 mg |
| Vitamin B12 | 50 mg |
| Vitamin A | 50 mg |
| Vitamin E | 50 mg |
| Vitamin C | 50 mg |

### Example 4 (Added to Example 3)

| | | |
|---|---|---|
| Molybdenum (aspartate) | 100 | mcg |
| Chromium (picolinate) | 200 | mcg |
| Boron | 3 | mg. |
| Vanadium | 90 | mcg. |
| Manganese (citrate) | 20 | mg. |
| Copper (citrate) | 2 | mg |
| Selenium (citrate) | 100 | mcg |

### Example 5

### Primary Chelator Blend

| | |
|---|---|
| Alpha lipoic acid | 10-60 mg |
| Cilantro Extract (25:1) | 200-600 mg |

### Example 6

### Primary/Secondary Chelator Blend

| | |
|---|---|
| Alpha lipoic acid | 10-250 mg |
| Quercetin | 10-250 mg |
| Cilantro Extract (25:1) | 200-600 mg |
| Glycine/methionine | 20-150 mg |

### Example 7

### Primary/Secondary Chelator Blend w Vascular System Promoter

| | |
|---|---|
| Alpha lipoic acid | 10-250 mg |
| Quercetin | 10-250 mg |
| Cilantro Extract (25:1) | 200-600 mg |
| Glycine/methionine | 20-150 mg |
| Ginkgo Biloba Extract | 10-250 mg |
| L-arginine | 20-200 mg |
| Rhodeola Rosea Extract | 50-250 mg |
| Green Tea Extract | 50-200 mg |

### Example 8

### Mineral Replacements—One or More Added to Examples 1-7

| | | |
|---|---|---|
| Calcium | 50-500 | mg |
| Magnesium | 20-200 | mg |
| Zinc | 5-200 | mg |
| Molybdenum | 10-100 | mcg |
| Chromium | 10-200 | mcg |
| Boron | 1-6 | mg. |
| Vanadium | 50-150 | mcg. |
| Manganese | 10-40 | mg. |
| Copper | 1-5 | mg |
| Selenium | 50-200 | mcg |

### Example 9

### Vitamins—One or More Added to Examples 1-8

| | | |
|---|---|---|
| Thiamine | 5-50 | mg |
| Vitamin B6 | 5-50 | mg |
| Folic acid | 5-50 | mg |
| Vitamin B12 | 5-50 | mg |
| Vitamin A | 100-1000 | IU |
| Vitamin E | 10-200 | IU |
| Vitamin C | 5-100 | mg |

### Example 10

| | | |
|---|---|---|
| Cilantro Extract | 250-500 | mg |
| Selenium | 50-200 | mg |
| Vitamin E | 10-200 | IU |
| Vitamin C | 10-100 | mg |
| Lipoic acid | 10-100 | mg |
| Glycine | 0-100 | mg |
| Cysteine | 0-100 | mg |
| Glutamate or glutamic acid | 0-100 | mg |
| Methionine | 0-100 | mg |

General

| Ingredient Ranges | Wt % |
|---|---|
| Alpha Lipoic Acid/Citantro Extract | 0-50 |
| Cilantro Extract | 0-50 |
| Quercetin/Gingko Biloba | 1-50 |
| Glycine/Methionine | 1-40 |
| L-arginine | 1-20 |
| Rhodeola Rosea Extract | 1-35 |
| Green Tea Extract | 1-35 |
| Calcium | 1-70 |
| Magnesium | 1-50 |
| Zinc | 1-10 |
| Thiamine | 1-5 |
| Vitamin B6 | 1-5 |
| Folic Acid | 1-5 |
| Vitamin B12 | 1-10 |
| Vitamin A | 1-5 |
| Vitamin E | 1-15 |
| Cysteine | 1-15 |
| Glutamate or glutamic acid | 1-15 |

Confectionary Formulations

The following are examples of gummy formulations that may be used for ease of oral administration of the compositions of the invention. The following is illustrative of various ranges for each ingredient, one or more of which, in any combination, can be incorporated into the confectionary formulation.

| Ingredient | Wt (g) |
|---|---|
| Cilantro Extract | 8.9 (range 4.0 to 12.0) |
| Gingko Extract | 4.4 (range 2.0 to 8.0) |
| Vascular System promoter | 4.4 (range 2.0 to 8.0) |
| Citric Acid Solution (10%) | 5.9 (range 0-10) |
| Beta Carotene | 2.0 (range 0-4.0) |
| *Sucrose | 146.5 |
| *Water | 43.9 and 46.9 |
| *42DE/A | 117.2 |
| *Gelatin | 23.4 |
| *Flavor | 0.98 |

*= base confectionary ingredients

400 g produces ≈200, 2 g gummy candies, with approximately 4 gummies per serving. The base for confectionary formulation may vary but typically includes sucrose, water and gelatin, but many other confectionary bases may be used.

Generally the confectionary base is the major weight component, with a formulated blend of ingredients integrated therewith. In other words, to a confectionary base may be included from 2 to 20% by weight of the inventive formulations. For example, each of examples 1-22 can be incorporated with a confectionary base, at from 2 to 20% by weight. Thus, various bases, such as hard candy, soft candy, chewable or powdered confectionaries may be formulated to deliver the formulations of the invention.

Using the dietary supplement of the invention limits the accumulation of heavy metals in the body, promotes removal of heavy metals previously accumulated in the body and alleviates the numerous symptoms and degenerative or neurocognitive conditions associated with heavy metal toxicity, as well as assists in reducing hyperactivity, chemical sensitivity, allergies fatigue, etc.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A composition for assisting natural body functions in cleansing the body of heavy metals, comprising:
cilantro, coriander or an extract thereof,
at least one primary chelator in an amount sufficient to move a heavy metal species from a user's central nervous system into the user's vascular system, and,
a vascular system promoter for promoting distribution of the primary chelator throughout the body.

2. The composition of claim 1 further comprising at least one secondary chelator or a precursor for stimulating or increasing production in the user's body of a secondary chelator for capturing said at least a portion of heavy metal species released from said primary chelator and to remove the captured heavy metal species from the body via an excretion pathway.

3. The composition of claim 1 wherein said primary chelator is selected from the group consisting of alpha lipoic acid, quercetin, gingko biloba, dihydroquercetin, rhamnetin, fisetin, dihydrofisetin, kaempferol, dihydrorubinetin, catechin, epicatechin, 3-hydroxyflavone, rutin (quercetin-3-rutinoside), quercetrin (quercetin-3-L-rhanmoside), hyperosid (quercetin-3-D-galactoside), robinin (kaempferol-3-rabinosie-7-L-rhamnoside, citrus bioflavanoid complex, lemon bioflavanoid complex, cyanadin, cyanadin chloride, esculetin, caffeic acid and combinations thereof.

4. The composition of claim 2 wherein said secondary chelator is a selected from the group consisting of quercetin, dihydroquercetin, rhanmetin, fisetin, kaempferol, 3-hydroxyflavone, catechin, epicatechin, rutin, quercitrin, hyperosid, robmm, glutathione, metallothionine, and combinations thereof and wherein said precursor is selected from the group consisting of glycine and methionine.

5. The composition of claim 1 wherein the vascular system promoter is selected from the group consisting of L-arginine, Rhodeola Rosea extract, citrulline, Ginkgo Biloba extract and combinations thereof.

6. The composition of claim 1, further comprising a replacement mineral selected from the group consisting of calcium, magnesium, zinc, molybdenum, chromium, boron, vanadium, manganese, copper, selenium and mixtures thereof.

7. The composition of claim 1, further comprising at least one vitamin or antioxidant selected from the group consisting of thiamine, vitamin B6, folic acid, vitamin B12, vitamin A, vitamin E, vitamin C, green tea extract, and combinations thereof.

8. The composition of claim 1 wherein said primary chelator is alpha lipoic acid, said vascular system promoter being a combination of Rhodeola Rosea extract and L-arginine.

9. The composition of claim 1 wherein said composition comprises Cilantro Extract (25:1), 200-600 mg, Gingko Biloba Extract (24/6)10-250 mg, Rhodeola Rosea Extract (0.8% salidrosides, 0.8% rosavins) 50-250 mg, Alpha lipoic acid 10-250 mg, and L-Arginine 20-200 mg.

10. The composition of claim 9 further comprising Vitamin C, 5-100 mg, Quercetin 10-250 mg, and Glycine/methionine 20-150 mg.

11. A composition for assisting natural body functions in cleansing the body of heavy metals, comprising:
cilantro, coriander or an extract thereof;
alpha lipoic acid in an amount sufficient to move amounts of a a heavy metal species from a user's central nervous system into the user's vascular system; and, optionally,
a secondary chelator in an amount sufficient for capturing at least a portion of said heavy metal species released from said primary chelator to limit return of said heavy metal species released back into the central nervous system; and
a vascular system promoter selected from the group consisting of L-arginine, Rhodeola Rosea extract, citrulline, Ginkgo Biloba extract and combinations thereof.

12. The composition of claim 11 wherein the alpha lipoic acid is present in an amount of about 10 mg to about 250 mg.

13. The composition of claim 13, wherein the cilantro, corriander or extract thereof is present in an amount of about 200 mg to about 600 mg.

14. A method of removing heavy metals from a mammal comprising
administering a composition containing cilantro, coriander or an extract thereof; at least one primary chelator in an amount sufficient to move a heavy metal species from a user's central nervous system into the user's vascular system, and, a vascular system promoter.

15. The method of claim 14 further comprising formulating the composition with a confectionary carrier for orally administering the composition.

16. The method of claim 15 further comprising adding to the confectionary carrier one or more of a component selected from the group consisting of a replacement mineral, a vitamin, a bioflavanoid, green tea extract, ginkgo biloba extract, an antioxidant, alpha lipoic acid, a tertiary chelator, glycine, methionine, and combinations thereof.
